(19) United States
(12) Patent Application Publication
(10) Pub. No.: US 2023/0233144 A1
(43) Pub. Date: Jul. 27, 2023

(54) Method for Determining Medication Adherence and Taggants Therefo

(71) Applicant: The Regents of the University of Colorado, A Body Corporate, Denver, CO (US)

(72) Inventors: Peter Anderson, Denver, CO (US); Lane Bushman, Aurora, CO (US)

(73) Assignee: The Regents of the University of Colorado, A Body Corporate, Denver, CO (US)

(21) Appl. No.: 18/175,418

(22) Filed: Feb. 27, 2023

Related U.S. Application Data
(63) Continuation of application No. PCT/US2021/047813, filed on Aug. 26, 2021.

(51) Int. Cl. A61B 5/00; A61K 47/22; G01N 33/94; G01N 33/58

(52) U.S. Cl. CPC A61B 5/4833; A61K 47/22; G01N 33/58; G01N 33/94; G01N 2458/15

(57) ABSTRACT

A method for direct measurement of gradients of patient compliance with a medication regimen by employing a stable non-radioactive isotope taggant compound as part of, bound to, or applied to an endogenous molecule. An assay to determine patient compliance with medication regimens for HIV pre-exposure prophylaxis employing administering a taggant to a pharmacologically active compound, obtaining and collecting a patient tissue or fluid sample analyzing the collected tissue or fluid sample for the taggant concentration and interpreting data from the taggant concentration analysis as an indicator of gradients of patient compliance.

### CROSS-REFERENCE TO RELATED APPLICATIONS

This application is a continuation of co-pending Patent Cooperation Treaty International Application Number PCT/US2021/047813 filed Aug. 26, 2021, from which priority is claimed.

### BACKGROUND OF THE INVENTION

Methods for measuring medication adherence can be categorized into two basic types: direct measurement and indirect measurement. Direct measurement refers to the firsthand observation of drug administration or the detection of the drug or its metabolite in a biological tissue or fluids, such as blood, urine, saliva, and/or hair samples. Direct methods are typically considered to be more accurate and informative than indirect methods; however, the complicated logistics of performing these measurements are an inherent disadvantage. As a result, indirect measurements (e.g. pharmacy refill records, pill counts, self-report, electronic medication vial-caps, etc.) are of a lower relative cost and greater ease for health care workers, but at the expense of accuracy and informative value.

Current pipeline products are being explored in order to provide direct measurements of medication adherence. The PROTEUS DISCOVER (Proteus Digital Health, Inc., Redwood City, California) is an ingestible marker useful in medication adherence and chronic disease management. A description of the Proteus ingestible marker and system may be found at U.S. Published Pat. Application No. 20200229758 published Jul. 23, 2020. The ID-CAP System by etectRx, Inc. (Gainesville, Florida) employs hard gelatin capsule with an imbedded ingestible wireless sensor that transmits a low power digit signal from within the patient’s gastrointestinal tract to an external wearable reader that, in turn, communicates data to applicant software resident on the patient’s smartphone. The ID-CAP system is described in, for example, U.S. Pat. 9047746, and is used for monitoring patient compliance with a medication regimen.

The present disclosure addresses the problem of medication non-adherence which is a significant health problem across a broad medical spectrum. The present disclosure employs a non-radioactive taggant added to medication which is then detectable in blood, urine, saliva and/or hair, such as by detection in dried blood spots obtained by finger prick. A particularly useful taggant is using 5+ adenine containing non-radioactive carbon13 (C13) and nitrogen 15 (N15), both of which are generally considered safe and are endogenous substances. Isotopes of hydrogen may also be employed as the non-radioactive stable taggant. Adenine and 5+adenine are taken up by red blood cells and phosphorylated to adenosine triphosphate (ATP and 5+ATP), which is highly abundant.

Proof of concept was conducted by an assay for a tenofovir anabolite (tenofovir-diphosphate, TFV-DP) in HIV pre-exposure prophylaxis (PrEP) with daily tenofovir-emtricitabine therapy showing strong correlations with PrEP outcomes. Studies with the non-radioactive taggant 5+ adenine show the same pharmacokinetic properties for 5+ ATP as TFV-DP. These assays identified TVF-DP and 5+ ATP in red blood cells in dried blood spots.

### SUMMARY OF THE INVENTION

It is an object of the present disclosure to provide a direct measurement method for determining medication adherence at relatively lower cost and increase ease of use as compared to indirect methods.

It is a further object of the present disclosure to provide a method for determining gradients of medication adherence.

It is another object of the present disclosure to provide a class of taggants useful in direct measurement methods for determining gradients of medication adherence.

It is still another object of the present disclosure to add a taggant to a medication and quantitating adherence to that tagged medication by mass spectrometry.

It is a further object of the present disclosure to use a stable-labeled, non-radioactive taggant of an endogenous molecule as the taggant.

It is yet another further object of the present disclosure that the non-radioactive taggant contain isotopes of hydrogen, carbon, nitrogen and/or oxygen.

It is still a further object of the present disclosure to use a taggant of an amino acid, a vitamin, a protein, and/or an enzyme as the taggant.

It is still a further object of the present disclosure to use 5+ adenine as the taggant.

It is still yet a further object of the present disclosure to use 5+ adenine as the taggant with adenosine triphosphate as the endogenous molecule to measure 5+ adenosine triphosphate in red blood cells.

It is yet still another object of the present disclosure to use hydroxyvitamin D3 and/or thyroxine as the taggant.

### BRIEF DESCRIPTION OF THE FIGURES

FIG. 1 is a proof of principle graph of the LCMS/MS ratio of 5+ adenosine triphosphate (5+ ATP) to +1 adenosine triphosphate (1+ATP) for daily administration of 5+ ATP to rats over six days.

FIG. 2 is a graph of tenofovir-diphosphate by dosing in human volunteers.

FIG. 3 is a table used in HIV PrEP trials illustrating adherence interpretations performed on the basis of tenofovir-diphosphate in dried blood spots.

FIG. 4 is a dose response graph of average taggant concentrations by dosing regimen from two studies in rats, showing gradients of adherence.

FIG. 5 is a graph of projected steady state concentrations by dosing regimen in rats, reflecting gradients of adherence.

FIG. 6 is a table illustrating adherence interpretations - analogous to TFV-DP -performed on the basis of taggant concentrations from the two rat studies.

FIG. 7 is a graph of taggant concentrations as a function of doses per week in humans.

### DETAILED DESCRIPTION OF THE PREFERRED EMBODIMENTS

The terminology used herein is for the purpose of describing example embodiments only and is not intended to be limiting. For purposes of clarity, the following terms used in this patent application will have the following meanings:

The term “about” is intended to mean a quantity, property, or value that is present at ±10%. Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range, including endpoints given for the ranges.is intended to mean an approximation of the value, shape or state referenced. For example, where used with a value, the term “about” is intended to include a variance of ±10% from the stated value, e.g., a stated value of 1 will also include the range of values between 0.9 and 1.1.

The term “medication adherence” is intended to mean the relative degree of a person’s compliance with a medication schedule and/or regimen.

The term “gradient,” when used in conjunction with “medication adherence” is intended to mean the relative metric of the person’s compliance with a medication schedule and/or regimen. For example, a low gradient medication adherence may indicate that the person is only infrequently taking a medication, whereas a high gradient medication adherence may indicate that the person is substantially complying with his/her medication schedule and/or regimen.

The term “substantially” is intended to mean a quantity, property, or value that is present to a great or significant extent and less than, more than or equal to totally. For example, substantially vertical may be less than, greater than, or equal to completely vertical.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

When an element or layer is referred to as being “on,” “engaged,” “connected,” or “coupled” to or with another element, it may be directly on, engaged, connected or coupled to the other element or layer, or intervening elements or layers may be present. In contrast, when an element is referred to as being “directly on,” “directly engaged to,” “directly connected to,” or “directly coupled to” or with another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Recitation of ranges of values herein are merely intended to serve as a shorthand method of referring individually to each separate value falling within the recited range, unless otherwise indicated herein, and each separate value is incorporated into the specification as if it were individually recited herein.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical, biomedical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

This detailed description of exemplary embodiments refers to the accompanying drawings, which show exemplary embodiments by way of illustration. While these exemplary embodiments are described in sufficient detail to enable those skilled in the art to practice the disclosure, it should be understood that other embodiments may be realized and that logical changes and adaptations in design and construction may be made in accordance with this disclosure and the teachings herein without departing from the spirit and scope of the disclosure. Thus, the detailed description herein is presented for purposes of illustration only and not of limitation.

In accordance with a preferred embodiment of the present disclosure, there is provided a method for direct measurement of patient compliance with a medication regimen. In accordance with another preferred embodiment of the present disclosure, there is provided a taggant compound which is used as a stable-label or tag bound to an endogenous molecule.

In accordance with the method of the present invention, an endogenous molecule, such as ATP, vitamin D, thyroxine, hypoxanthine, uracil, creatine, pyridoxine or the like is labeled with a stable isotope non-radioactive tag, synonymously referred to herein as a taggant. The taggant may, for example, be adenine 5+, adenine with a total of five C13 and or N15 atoms. The taggant adenine 5+ is metabolized to ATP yielding 5+ adenosine triphosphate, which is differentiable by mass spectrometry from the endogenous ATP having non-isotopic C12 and N14 in the adenine component of endogenous ATP.

Stable isotopes of hydrogen (¹H, ²H, and ³H) carbon (C13), nitrogen (N15) and oxygen (O16, O17) are naturally occurring and are known to be useful as research tools in conjunction with mass spectrometry in studies of bioavailability and release kinetics of drugs. Schellekens, R., et al., Applications of stable isotopes in clinical pharmacology, Br. J. Clin. Pharmacol., 72:6, 2011, 879-897.

Example 1: A method for estimating patient adherence to a medication regime was investigated by administering 100 mcg of 5+ ATP (the taggant being adenine with five C13 and/or N15 atoms) to rats daily for six days. Once each day, blood spot samples were taken from each rat and dried. The levels of 5+ ATP were measured in the red blood cells in the dried blood spot samples by mass spectrometry. The levels of the 5+ ATP were measured, and the mass spectrometry signal was compared to naturally occurring 1+ ATP, which occurs in about 1% of the total ATP pool. The ratio of the 5+ATP to the 1+ATP signals was then calculated and half-life was determined. The half-life was in the range to provide a measure of how much - in gradients - of 5+ adenine was ingested over the preceding month, thereby representing the average adherence over the prior month.

The in vivo ATP pool is large, the half-life of ATP is relatively long, 3 days in rats, corresponding to about 10 days in humans. Therefore, the ratio of the 5+ ATP to 1+ ATP is a predictive measurement of the average adherence over the prior month period which is directly representative of the amount of 5+ adenine was ingested over that preceding month. Given the long half-life of ATP, the measurement for gradients of adherence is analogous to hemoglobin A1C as a measure of gradients of glucose exposure over the preceding months.

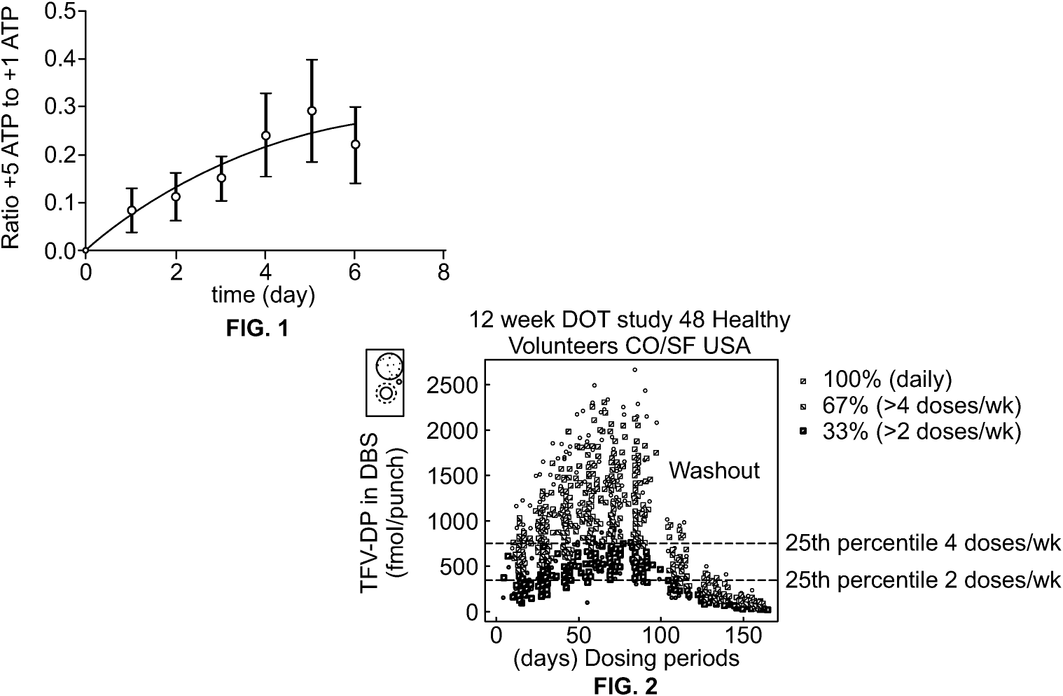

FIG. 1 is a graph of the ratio of +5 ATP to +1 ATP over time in days in rats as measured in dried blood spots obtained from tail nicks. It can be seen that the ratio of +5 ATP to +1 ATP increases in a predictable manner over the six day period measured.

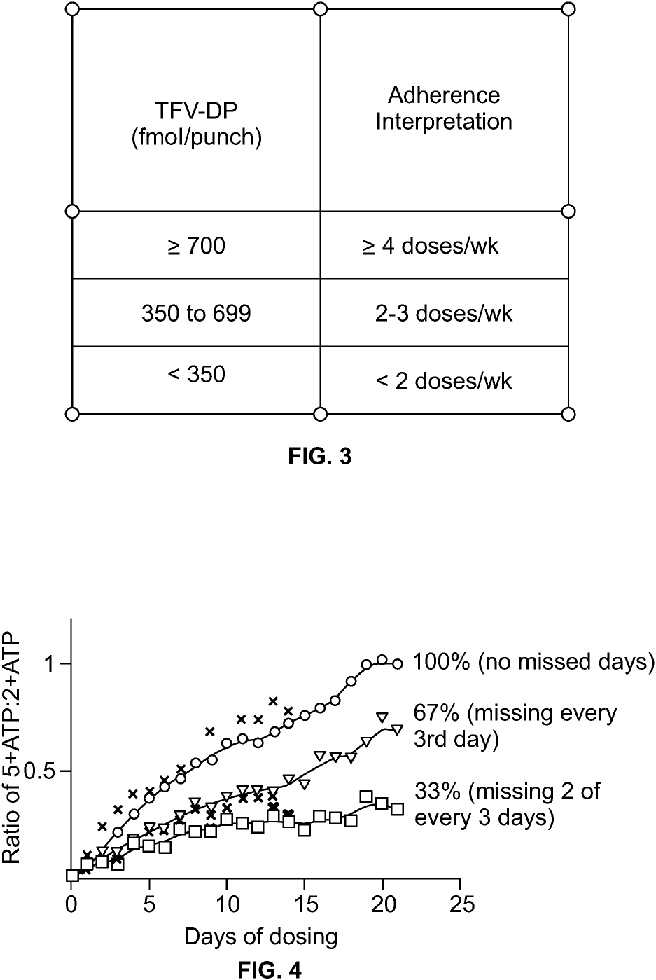

Example 2: An assay for a tenofovir (a PrEP drug) anabolite, tenofovir-diphosphate in red blood cells, which may be collected in dried blood spots (DBS), is disclosed as an adherence assessment. TFV-DP builds up to high levels in red blood cells with consistent adherence because it exhibits a 17 day half-life and 25-fold accumulation from a single dose to steady-state. This enables assessment of cumulative dosing, indicative of degrees or gradients of adherence, over the preceding 1-2 months for tenofovir-based PrEP. This is analogous to how hemoglobin A1C works for cumulative glucose exposure. FIG. 2 shows a pharmacokinetic study in humans (n=48) conducted using directly observed PrEP dosing (DOT) of 2.3 doses/week (33% of daily), 4.7 doses/week (67% of daily), and 7 doses/week (100%). FIG. 2 illustrates the “build up” of TFV-DP in DBS over approximately 60 days until it reaches a steady-state plateau, followed by washout. As can be seen, the drug concentrations can distinguish low, medium, and high adherence (2 doses/week, 4 doses/week and daily dosing). FIG. 3 illustrates how taggant tenofovir diphosphate concentration in dried blood spots is used in clinical trials to correlate and interpret medication adherence based upon TFV-DP concentration in DBS.

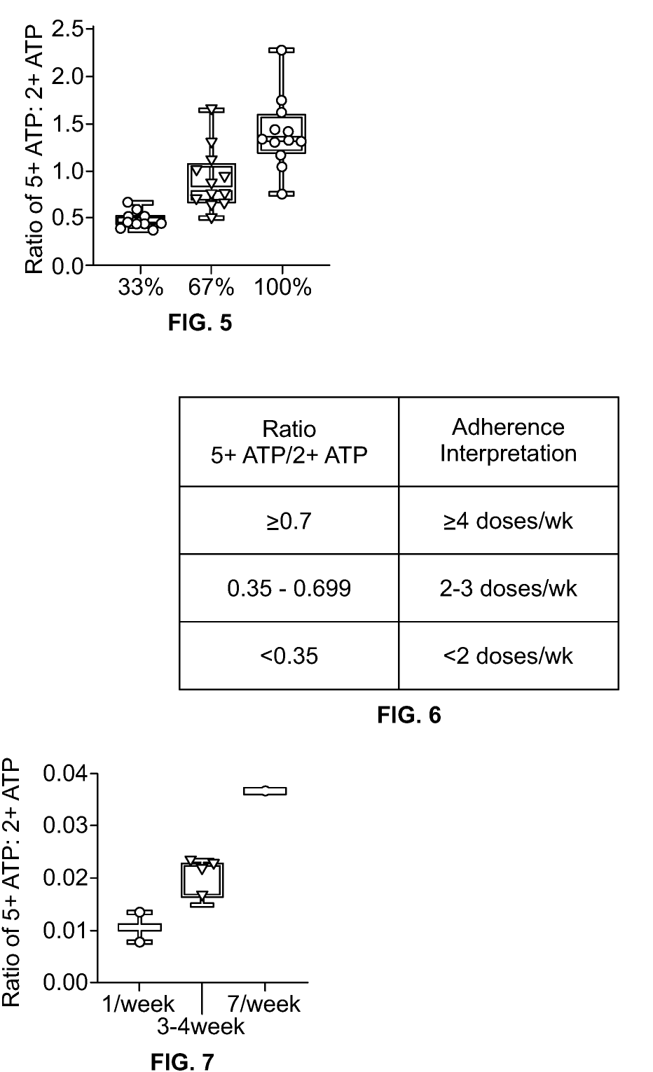

Example 3: To show further proof of concept, oral doses of 0.2 mg of 5+ adenine with C13 and/or N15 were given to thirty-six rats (IACUC protocol 00234). The rats were randomized to 0, 2.3 (33%), 4.7 (67%), or 7 (100%) doses per week for 14 days (study 1) and 21 days (study 2), with a washout separating the studies. Doses were delivered orally in peanut butter to replicate normal ingestion. Daily blood was collected via tail-nick as dried blood spots (DBS). The ratio of taggant (5+ ATP) to naturally-occurring 2+ ATP was measured from DBS using mass spectrometry. FIG. 3 shows the averaged concentrations for studies 1 and 2 and FIG. 5 shows the individual steady state projections using standard first-order pharmacokinetic calculations. The washout half-life was approximately 10 days (not shown). From these findings, the adherence table of FIG. 6 was established for the ratio of 5+ ATP at a taggant to native 2+ ATP. This adherence table is analogous to that of TFV-DP, which has proven useful in clinical trials.

Example 4: Finally, an ongoing pilot of 5+ adenine in human volunteers (COMIRB 02-0332) has been conducted with consulting adults randomized to one of 2 sequences consisting of two directly observed dosing (DOT) regimens with 2 mg adenine 5+ per dose, 1 dose/week followed by 4 doses/week or 3 doses/week followed by 7 doses/week. Each dose regimen lasted approximately 12 weeks and was separated by approximately a 12-week washout period for a total study duration of approximately 36 weeks. To the date of this application, six subjects have contributed data. FIG. 7 shows last concentrations collected during dosing, ranging from week 6 to 11 for the six subjects.

The human study demonstrates that the concentrations follow the same profile shown in the rat studies discussed above, suggesting the taggant works similarly in humans. Moreover, the small 2 mg dose is very small and fits the profile of a taggant that could be added to any medication or placebo. For the human study, the taggant was formulated into a small capsule with an inert excipient.

In view of the foregoing experimental results, similar non-radioactive stable isotope taggants may be added to a virtually any medication and allow for blood, saliva, urine, and/or hair tests to assess average adherence to the labeled medication over the preceding month. By using the naturally occurring pool of 1+ or 2+ ATP, the ratio of taggant labeled ATP to 1+ or 2+ ATP allows the ratio to be individualized to each patient because the endogenous molecule is being used to normalize the values of the taggant. Further, by employing low doses of taggant, e.g., between about 0.1 mg and about 50 mg, the taggant may be sprayed or otherwise applied to medications or it may, alternatively be added to excipients.

The data strongly suggests that a stable-labeled isotope endogenous substance may be used as a taggant that is capable of addition to any medication or placebo to quantify adherence. By employing this taggant, better understanding of drug efficacy based upon actual drug ingestion, i.e., adherence, can be achieved in clinical trials and in clinical practice to understand patient compliance with drug regimens.

In accordance with the foregoing description, it will be appreciated that there is described a method for determining medication adherence, comprising the step of administering to a patient in need thereof a medication having a stable non-radioactive taggant added to the medication; obtaining a sample from the patient; assaying the sample for a concentration of the non-radioactive taggant in the sample. Wherein the stable non-radioactive taggant may be added to the medication as an excipient, as a spray, suspension, or other means by which the medication and the taggant are co-administered. The taggant, which may have a non-radioactive isotope of hydrogen, carbon, nitrogen, and/or oxygen, may be added to an endogenous molecule, such as for example, an amino acid, a vitamin, a protein, and/or an enzyme. The endogenous molecule may be selected from the group consisting of ATP, vitamin D, thyroxine, hypoxanthine, uracil, creatine, and pyridoxine.

It will also be understood that the present disclosure presents a medicament comprising a non-radioactive taggant, such as 5+ adenine, in combination with HIV pre-exposure prophylaxis medications.

These and other aspects of the invention will be understood by those skilled in the art to not be limited to the foregoing detailed description, dosages, concentrations, compounds, taggants, or detection methodologies. Rather, the scope of the present invention is intended to be limited and defined only by the claims appended hereto.

What is claimed is:

1. A method for determining medication adherence, comprising the step of administering to a patient in need thereof a medication having a stable non-radioactive taggant added to the medication; obtaining a sample from the patient; assaying the sample for a concentration of the non-radioactive taggant in the sample.

2. The method of claim 1, wherein the stable non-radioactive taggant is added to the medication as an excipient.

3. The method of claim 1, wherein the stable non-radioactive taggant is added to the medication as a spray.

4. The method of claim 1, wherein the stable non-radioactive taggant is added to the medication at a dosage level between about 0.1 mg to about 50 mg.

5. The method of claim 1, wherein the stable non-radioactive taggant further comprises at least one of a stable isotope of carbon, nitrogen or oxygen.

6. The method of claims 1, wherein the stable non-radioactive taggant is added to an endogenous molecule.

7. The method of claim 6, wherein the stable non-radioactive taggant is added to an amino acid.

8. The method of claim 6, wherein the stable non-radioactive taggant is added to a protein.

9. The method of claim 6, wherein the stable non-radioactive taggant is added to an enzyme.

10. The method of claim 6, wherein the stable non-radioactive taggant is added to a vitamin.

11. The method of claim 6, wherein the endogenous molecule is selected from the group consisting of adenine, vitamin D, thyroxine, hypoxanthine, uracil, creatine, and pyridoxine.

12. The method of claim 1, wherein the medication further comprises an HIV pre-exposure prophylaxis medication.

14. A medicament comprising a pharmacologically active compound in combination with a non-radioactive stable taggant.

15. The medicament according to claim 14, wherein the non-radioactive taggant further comprises 5+ adenine.

16. The medicament of claim 15, wherein the 5+ adenine taggant is added as an excipient.

17. The medicament of claim 15, wherein the 5+ adenine taggant is added as a spray.

18. The medicament of claim 15, wherein the 5+ adenine is present at a dosage level between about 0.1 mg to about 50 mg.

19. The medicament of claim 15, wherein the 5+ adenine includes a non-radioactive isotope of carbon, nitrogen and/or oxygen.
